(19) United States
(12) Patent Application Publication
(10) Pub. No.: US 2015/0208951 A1
(43) Pub. Date: Jul. 30, 2015

(54) BREATH SAMPLING TUBES

(71) Applicant: ORIDION MEDICAL 1987 LTD., Jerusalem (IL)

(72) Inventor: Paul S. Addison, Midlothian (GB)

(73) Assignee: ORIDION MEDICAL 1987 LTD., Jerusalem (IL)

(21) Appl. No.: 14/167,745

(22) Filed: Jan. 29, 2014

(51) Int. Cl. A61B 5/097; A61B 5/08; A61M 16/04

(52) U.S. Cl. CPC A61B 5/097; A61M 16/0488; A61B 5/082

(57) ABSTRACT

The present disclosure provides breath sampling tubes having an outer wall and an inner wall, wherein the outer wall includes at least one groove and wherein at least part of the tube is formed of a material configured to evaporate liquid.

### TECHNICAL FIELD

The present disclosure generally relates to the field of breath sampling tubes.

### BACKGROUND

Accurate monitoring concentrations of a gas, such as for example carbon dioxide (CO₂) in exhaled breath is vital in assessing the physiologic status of a patient. Breath sampling is generally performed through breath sampling tubes configured to be connected to a patient airway and to a medical device.

Liquids are common in patient sampling systems, and have several origins, for example condensed out liquids from the highly humidified air provided to and exhaled from the patient. These liquids typically accumulate both in the patient airway and in the sampling line tubing; secretions from the patient, typically found in the patient airway; and medications or saline solution provided to the patient during lavage, suction and nebulization procedures.

### SUMMARY

The present disclosure relates to breath sampling tubes including an outer wall having at least one groove. The breath sampling tubes disclosed herein are configured to evaporate liquids.

One of the major obstacles when designing a filter system is the necessity to prevent any liquids from blocking the breath sampling path or from reaching the measurement sensor while providing continuous, smooth, undisturbed sampling of the patient's breath.

A well-known problem with gas sampling lines is that they may eventually saturate allowing the line to become clogged. Lines are designed so that water vapor is captured and evaporated through the tube surface. At high humidity, however, the evaporation flow rate may be less than the capture rate and so eventually the reservoir, or other suitable liquid collection element, saturates and the line may become clogged. The time it takes for this to happen is known as the lifetime of the line.

The breath sampling tubes disclosed herein, include an outer wall and an inner wall, wherein at least a part of the outer wall has at least one groove. At least a part of the breath sampling tube is made from a material configured to evaporate there through liquids. The at least one groove in the outer wall increases the surface area of the outer wall. By increasing the surface area of the outer wall the evaporation of liquids from the tube surface is enhanced and hence the lifetime of the line extended.

The groove(s) may be formed parallel to the main axis of the tube (i.e. longitudinally). Alternatively the groove(s) may be formed orthogonal to this axis, circumferentially around the tube. Alternatively, the groove(s) may be formed helically around the tube. The formation of the groove(s) longitudinally, circumferentially or helically around the tube may further serve to influence the rigidity of the tube and the ease of manufacturing.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

According to some embodiments, there is provided a breath sampling tube having an outer wall and an inner wall, wherein the outer wall includes at least one groove and wherein at least part of the tube is formed of a material configured to evaporate liquid.

According to some embodiments, the at least one groove extends substantially along the length of said tube.

According to some embodiments, the at least one groove is formed parallel to a main axis of the breath sampling tube. According to some embodiments, the at least one groove is formed orthogonal to a main axis of the breath sampling tube.

According to some embodiments, the at least one groove is formed helically to a main axis of the breath sampling tube. According to some embodiments, the at least one groove is formed unevenly on the outer wall.

According to some embodiments, the outer wall includes a plurality of grooves.

According to some embodiments, the at least one groove generates a rough surface in the outer wall.

According to some embodiments, the at least one groove increases the surface area of the outer wall. According to some embodiments, the at least one groove enhances the evaporation of liquids from the breath sampling tube, thereby extending the life time of the breath sampling tube.

According to some embodiments, the material configured to evaporate liquids is a hydrophilic material. According to some embodiments, the outer wall includes a hydrophilic material. According to some embodiments, the inner wall includes a hydrophilic material.

According to some embodiments, the tube further includes an inner conduit. According to some embodiments, the inner conduit is configured to permit gas flow along a central portion of the conduit and to store liquids along a surface of the conduit.

According to some embodiments, the surface of the inner conduit includes a hydrophilic material.

According to some embodiments, there is provided a breath sampling system including: a breath sampling tube having an outer wall and an inner wall wherein at least part of the outer wall includes at least one groove and wherein at least part of the tube is formed of a material configured to evaporate liquids; and at least one connector.

According to some embodiments, there is provided a method including forming a breath sampling tube having an outer wall and an inner wall, wherein at least a part of the outer wall includes at least one groove.

According to some embodiments, at least part of the tube is formed of a material configured to evaporate water.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1A schematically illustrates evaporation through a surface of a tube, according to some embodiments;

FIG. 1B schematically illustrates the lifetime of tube lines against the vapor pressure of water (P_{H2O}) in prior art tube lines (A), and for the tube lines disclosed herein (B), according to some embodiments;

FIG. 2A schematically illustrates a tube with grooves longitudinally to the tubing line, according to some embodiments;

FIG. 2B schematically illustrates a tube with grooves circumferentially around the tubing line, according to some embodiments;

FIG. 2C schematically illustrates a tube with grooves helically around the tubing line, according to some embodiments;

FIG. 3A schematically illustrates a tube with grooves in the entire length thereof, according to some embodiments;

FIG. 3B schematically illustrates a tube with grooves at a distal end thereof, according to some embodiments;

FIG. 3C schematically illustrates a tube with grooves at a proximal end thereof, according to some embodiments;

FIG. 3D schematically illustrates a tube with grooves at a central portion thereof, according to some embodiments;

FIG. 3E schematically illustrates a tube with grooved sections along the tubing line, according to some embodiments;

FIG. 4A schematically illustrates a tube with grooves and with an inner conduit, according to some embodiments;

FIG. 4B schematically illustrates a tube with grooves and with an inner conduit, according to some embodiments;

FIG. 5 schematically illustrates a breath sampling system, according to some embodiments.

FIG. 6 schematically illustrates a breath sampling system, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

There is provided, according to some embodiments, a tube including an outer wall and an inner wall, wherein at least a part of the outer wall includes at least one groove. According to some embodiments, at least part of the tube is formed of a material configured to evaporate fluids.

According to some embodiments, the tube is a breath sampling tube. According to some embodiments, the tube is part of a breath sampling tube. According to some embodiments, the tube is configured to be connected to a breath sampling tube. The tubes disclosed herein may be integrally formed with a commonly used breath sampling tube or be a separate element (and/or an “add-on”) which may be attached to a breath sampling tube, for examples by adaptor(s) and or connector(s).

As used herein, the terms “breath sampling tube”, “sampling line” and “breath sampling line” may refer to any type of tubing(s) or any part of tubing system adapted to allow the flow of sampled breath, for example, to an analyzer, such as a capnograph. The sampling line may include tubes of various diameters, adaptors, connectors, valves, drying elements (such as filters, traps, trying tubes, such as Nafion® and the like).

As used herein, the term “at least a part of” may refer to the entire tube, the proximal end of the tube, the distal end of the tube, a central part of the tube, in proximity to a liquid trap or reservoir, at a certain distance from a liquid trap or reservoir, as sections along the tube or any other suitable part of the tube line. Each possibility is a separate embodiment.

As used herein, the terms “distal” and “distal end” may refer to the part of the tube closest to the subject. The length of the distal end may for example be 0.5, 1, 2, 3, 4, 5, 10 cm or more. Each possibility is a separate embodiment.

As used herein, the terms “proximal” and “proximal end” may refer to the part of the tube closest to the medical device. The length of the proximal end may for example be 0.5, 1, 2, 3, 4, 5, 10 cm or more. Each possibility is a separate embodiment.

As used herein, the term “close proximity” may refer to 30, 20, 15, 10, 5, 1, 0.5 cm or less. Each possibility is a separate embodiment.

As used herein, the term “certain distance” may refer to a distance larger than 10 cm, for example larger than 20 cm, 30 cm, 40 cm or 50 cm, 70 cm. Each possibility is a separate embodiment.

As used herein, the term “groove(s)” may refer to a channel or a furrow formed in the outer wall of the breath sampling tube.

As used herein, the term “at least one groove” may refer to one groove, 2 grooves, 3 grooves, 4 grooves, 5 grooves, 10 grooves, 100 grooves or more, any number there between or any other suitable number of grooves. Each possibility is a separate embodiment. For example, according to some embodiments, the outer wall comprises at least 3 grooves. For example, according to some embodiments, the outer wall comprises at least 10 grooves.

According to some embodiments, the outer wall comprises a plurality of grooves. According to some embodiments, the plurality of grooves generates a rough surface in the outer wall.

According to some embodiments, the groove(s) increase the surface area of the outer wall. It is understood by one of ordinary skill in the art, that increasing the surface area of the tube facilitates enhanced evaporation of liquids from the tube and hence extends the life time of the tube. Therefore, under normal vapor pressure of water (P_{H2O}) conditions, the tube of the present disclosure can evaporate more liquids, thereby avoid blockage of the tube and in effect extend its lifetime.

According to some embodiments, the groove(s) may be replaced by a ridge(s)/elevation(s), which likewise serves to increase the surface area of the tube, and as such fall under the scope of this disclosure.

According to some embodiments, the asymmetric grooves may be consecutive along or around the tube, such that two successive grooves share a wall. Alternatively, the asymmetric grooves may not be immediately successive but rather be separated, such that each groove has it separate walls. For example, each groove may be separated at least 1 mm, at least 5 mm, at least 1 cm, at least 5 cm or more from its closest neighboring groove. Each possibility is a separate embodiment.

According to some embodiments, the material configured to evaporate liquids is a hydrophilic material. According to some embodiments, the outer wall includes a hydrophilic material. According to some embodiments, the inner wall includes a hydrophilic material. According to some embodiments, the hydrophilic material is a hydrophilic wicking material such as a porous plastic having a pore size ranging from approximately 5 microns to approximately 50 microns.

According to some embodiments, the at least one groove may be formed parallel to a main axis of a tube (longitudinally), such as for example a breath sampling tube. Alternatively, according to some embodiments, the at least one groove may be formed orthogonal to a main axis of the tube (circumferentially). Alternatively, according to some embodiments, the at least one groove may be formed helically to a main axis of the tube. Alternatively, according to some embodiments, the at least one groove may be formed unevenly on the outer wall of the tube. It is understood by one of ordinary skill in the art, that the pattern of the groove(s) on the tube may influence the flexibility of the tube. For example, circumferential groove(s) or helical groove(s) around the tube may form a tube with greater flexibility as compared to a tube with longitudinal groove(s), or as compared to a tube without groove(s). Such flexibility may be important both when in use and for more efficient packaging and storage.

According to some embodiments, the tube further comprises an inner conduit. According to some embodiments, at least a portion of the inner conduit is non-cylindrical and configured to store liquids. According to some embodiments, the inner conduit is configured to permit gas flow along a central portion of the conduit and to store liquids along a surface of the conduit. According to some embodiments, the surface of the inner conduit comprises a hydrophilic material. According to some embodiments, the inner conduit may include a first lumen and a second lumen. According to some embodiments, the diameter of the first lumen is larger than the diameter of the second lumen. According to some embodiments, the inner conduit may be adapted to collect liquids in the first lumen and to permit gas flow in the second lumen. According to some embodiments, the surrounding surface of the first lumen may be more hydrophilic than the surrounding surface of the second lumen.

There is provided, according to some embodiments, a breath sampling system comprising: a breath sampling tube comprising an outer wall and an inner wall wherein at least part of the outer wall comprises at least one groove and wherein at least part of the tube is made from a material configured to evaporate liquids; and at least one connector. It is understood that the tube of the breath sampling system may be the tube described in any one or more of the above embodiments. According to some embodiments, the connector is molded on the breath sampling tube. According to some embodiments, the connector is a separate element configured to be attached to the breath sampling tube.

According to some embodiments, the connector is configured to connect between the breath sampling tube and a patient airway tubing. According to some embodiments, the connector is configured to connect to an oral/nasal cannula.

According to some embodiments, the system further comprises a moisture reduction system, hereinafter referred to as MRS. The MRS may be a specially designed tube, which may be of variable length and diameter, adapted to reduce moisture entering the breath sampling tube. The MRS may include any drying mechanism and/or material, essentially impermeable to gas, that is capable of reducing moisture level, such as but not limited to a Nafion® tube. According to some embodiments, the system may further include filters such as micro-porous filters or molecular sieves (material containing tiny pores of a precise and uniform size that may be used to absorb moisture). According to some embodiments, the system may further include a liquid trap and/or reservoir configured to trap liquids in the sampling tube. According to some embodiments the system may further comprise a medical device such as but not limited to a capnograph.

There is provided, according to some embodiments, a breath sampling system comprising: a breath sampling tube comprising an outer wall and an inner wall wherein at least part of the outer wall comprises at least one groove and wherein at least part of the tube is made from a material configured to evaporate liquids; and an oral nasal cannula. It is understood that the tube of the breath sampling system may be the tube described in any one or more of the above embodiments. According to some embodiments, the oral/nasal cannula is an integral part of the breath sampling tube. According to some embodiments, the oral/nasal cannula is molded on the breath sampling tube. According to some embodiments, the oral/nasal cannula is a separate element configured to be attached to the breath sampling tube. According to some embodiments, the system further comprises a moisture reduction system, as essentially described above.

According to some embodiments, there is provided a method comprising forming a breath sampling tube having an outer wall and an inner wall, wherein at least a part of the outer wall comprises at least one groove. According to some embodiments, at least part of the tube is formed of a material configured to evaporate liquids. It is understood that the at least one groove may have any distribution and/or configuration along and/or around the sampling tube. For example the at least one groove may be formed circumferentially, longitudinally or helically in the outer wall of the tube, as essentially described above. For example the at least one groove may be formed along the entire length of the tube or in parts thereof, as essentially described above. Furthermore the number of grooves made may be any suitable number, as essentially described above. According to some embodiments, the at least one groove may be replaced by at least one ridge or elevation, which likewise serves to increase the surface area of the tube, and as such fall under the scope of this disclosure.

There is provided, according to some embodiments, a method for breath sampling comprising: channeling breath through a breath sampling tube, the breath sampling tube comprising an outer wall and an inner wall wherein at least part of the outer wall comprises at least one groove and wherein at least part of the tube is made from a material configured to evaporate liquids. It is understood that the tube of the method may be the tube described in any one or more of the above embodiments.

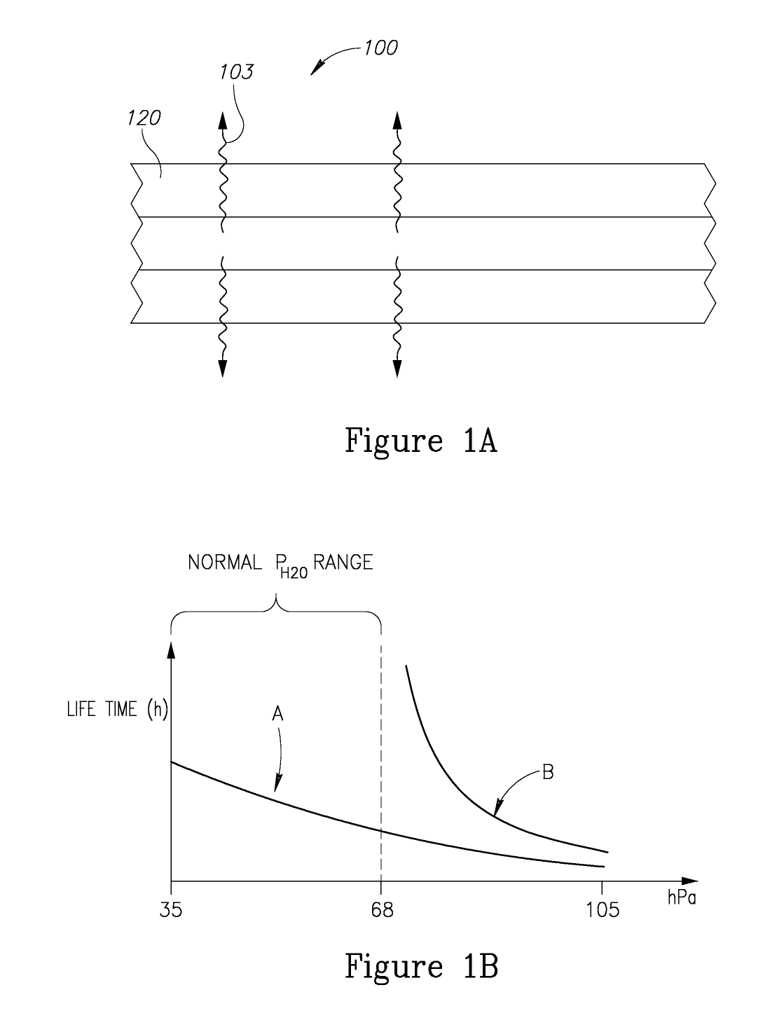

Reference is now made to FIG. 1A, which schematically illustrates evaporation through a surface of a tube 100, according to some embodiments. Tube 100, may for example be a breath sampling tube, and is generally configured to allow water to evaporate (shown as arrows 103) through an outer wall 120 of tube 100. According to some embodiments, outer wall 120 is made of a hydrophilic material, such as for example a hydrophilic wicking material such as a porous plastic having a pore size ranging from approximately 5 microns to approximately 50 microns.

FIG. 1B schematically illustrates the lifetime of tube lines against the vapor pressure of water (P_{H2O}) in prior art tube lines (A), and in the tube lines disclosed herein (B), according to some embodiments. It is understood that as the vapor pressure (humidity) increases the life time of the sampling tube decreases. The normal P_{H2O} typically ranges from about 35 hPa to about 70 hPa, however P_{H2O} values as high as 105 hPa can also occur.

As seen in FIG. 1B, the tube disclosed herein, comprising a groove(s) in an outer wall thereof, has an extended life time due to the enhanced evaporation achieved through the wall of the tube.

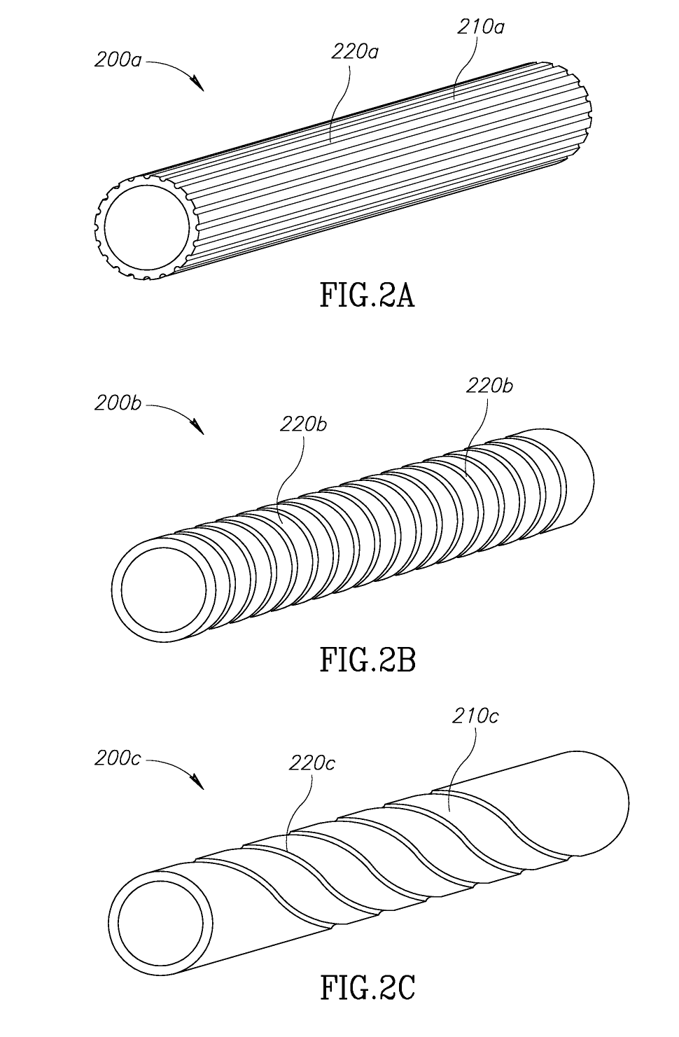

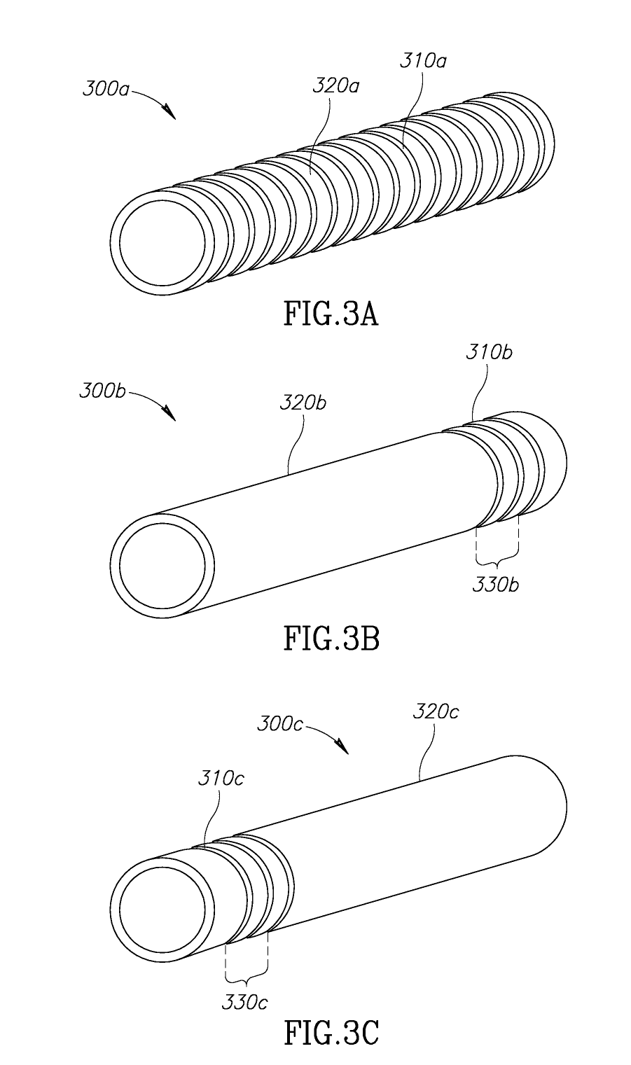

FIG. 2A-C schematically illustrates longitudinal views of parts of breath sampling tubes with a groove(s), according to some embodiments. It is understood that groove(s) depicted are representative only and that the number of grooves and/or their distribution along and/or around the sampling tube may vary, for example the grooves may be successive or non-successive, be distributed circumferentially, longitudinally or helically along the entire length of the tube or parts thereof. Such variations in the number and/or distribution of the grooves fall under the scope of this disclosure. It is further clear, that as the number of grooves increases, each groove becomes less distinct and the outer wall of the tubes obtains an overall rough appearing surface. Furthermore, according to some embodiments, the groove(s) may be replaced by a ridge(s) or an elevation(s) in the outer wall, which likewise serves to increase the surface area of the tube, and as such fall under the scope of this disclosure.

FIG. 2A schematically illustrates a breath sampling tube 200a with grooves 210a along an outer wall 220a, according to some embodiments.

FIG. 2B schematically illustrates a breath sampling tube 200b with grooves 210b circumferentially distributed in an outer wall 220b of tube 200b, according to some embodiments.

FIG. 2C schematically illustrates a breath sampling tube 200c with a groove 210c helically distributed in an outer wall 220c of tube 200c, according to some embodiments.

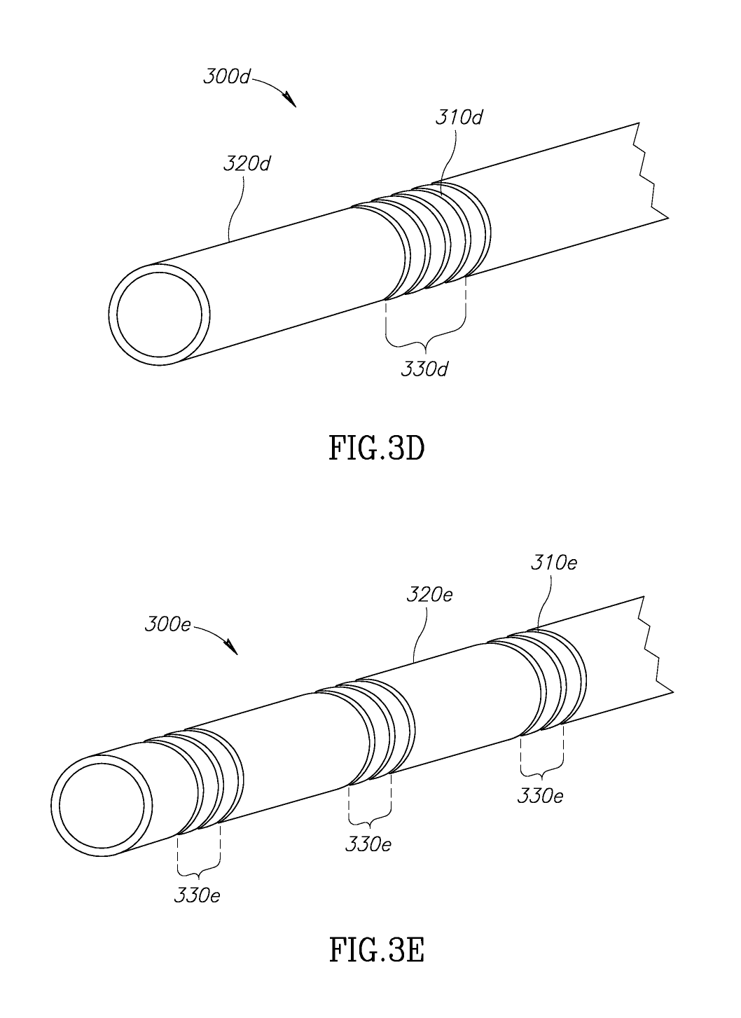

FIG. 3A-E schematically illustrates longitudinal views of parts of breath sampling tubes with a groove(s), according to some embodiments. It is understood that the grooves depicted in FIG. 3A-E as circumferentially distributed in the outer wall around the tube are representative only and that the number of grooves, their distribution and/or configuration along and/or around the sampling tube may vary, for example the grooves may be successive or non-successive, be distributed circumferentially, longitudinally or helically along the entire length of the tube or parts thereof. Such variations in the number, configuration and/or distribution of the grooves fall under the scope of this disclosure. It is further clear, that as the number of grooves increases, each groove becomes less distinct and the outer wall of the tubes obtains an overall rough appearing surface. Furthermore, according to some embodiments, the grooves may be replaced by ridges or elevations in the outer wall, which likewise serves to increase the surface area of the tube, and as such fall under the scope of this disclosure.

FIG. 3A schematically illustrates a breath sampling tube 300a with grooves 310a in an outer wall 320a along the entire length of tube 300a, according to some embodiments.

FIG. 3B schematically illustrates a breath sampling tube 300b with grooves 310b in an outer wall 320b at a distal end 330b of tube 300b, according to some embodiments. It is understood that distal end 330b depicted is non-limiting and serves an illustrative purpose only. Distal end 330b may be short, extending a few centimeters, such as for example 2, 3, 4, 5 cm from the end of tube 300b, or longer such as for example 10, 15, 20, 25, 30 cm or more from the end of tube 300b. Each possibility is a separate embodiment.

FIG. 3C schematically illustrates a breath sampling tube 300c with grooves 310c in an outer wall 320c at a proximal end 330c of tube 300c, according to some embodiments. It is understood that proximal end 330c depicted is non-limiting and serves an illustrative purpose only. Proximal end 330c may be short, extending a few centimeters, such as for example 2, 3, 4, 5 cm from the end of tube 300c, or longer such as for example 10, 15, 20, 25, 30 cm or more from the end. Each possibility is a separate embodiment.

FIG. 3D schematically illustrates a breath sampling tube 300d with grooves 310d in an outer wall 320d at a central portion 330d thereof, according to some embodiments. It is understood that central portion 330d depicted is non-limiting and serves an illustrative purpose only. Central portion 330d may be short, extending a few centimeters, such as for example 2, 3, 4, 5 cm or longer such as for example 10, 15, 20, 25, 30 cm or more. Each possibility is a separate embodiment.

FIG. 3E schematically illustrates a breath sampling tube 300e sections 330e having grooves 310e in an outer wall 320e, according to some embodiments. It is understood that sections 330e depicted are non-limiting and serve an illustrative purpose only. The length of each section 330e may be short, extending a few centimeters, such as for example 2, 3, 4, 5 cm or longer such as for example 10, 15, 20, 25, 30 cm or more. Each possibility is a separate embodiment. Furthermore, the number of sections 330e may vary from a single section to 2, 3, 4, 5, 10 or more sections. Each possibility is a separate embodiment. According to some embodiments, each tube comprises a plurality of sections.

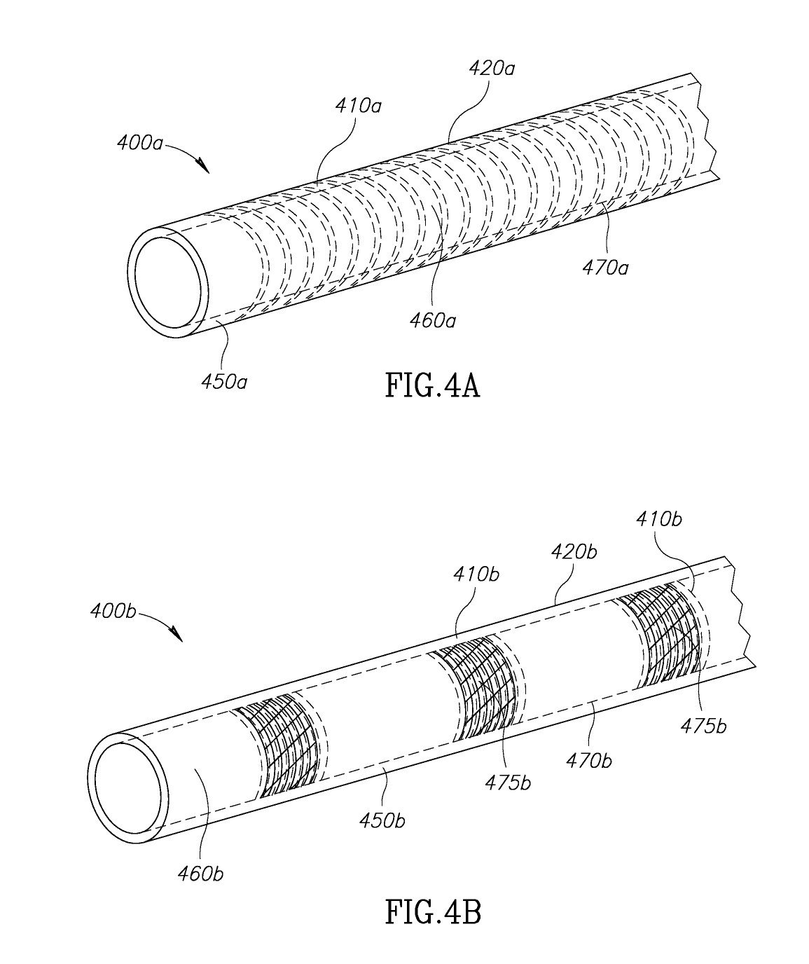

FIG. 4A-B schematically illustrates breath sampling tubes with a groove(s) in an outer wall thereof, and with an inner conduit. It is understood that the grooves in FIG. 4A-B may be of any configuration and distribution and that the number of grooves may vary, for example the grooves may be successive or non-successive, be distributed circumferentially, longitudinally or helically along the entire length of the tube or parts thereof. Such variations in the number, configuration and/or distribution of the grooves fall under the scope of this disclosure. It is further clear, that as the number of grooves increases, each groove becomes less distinct and the outer wall of the tubes obtains an overall rough appearing surface. Furthermore, according to some embodiments, the grooves may be replaced by ridges or elevations in the outer wall, which likewise serves to increase the surface area of the tube, and as such fall under the scope of this disclosure.

FIG. 4A schematically illustrates a breath sampling tube 400a with grooves 410a along the entire length of an outer wall 420a and with an inner conduit 450a, according to some embodiments. Inner conduit 450a may be configured to permit gas flow along a central portion 460a of conduit 450a and to store liquids along a surface 470a of conduit 450a. Optionally, surface 470a of inner conduit 450a include a hydrophilic material. The liquids flowing in inner conduit 450a are then repelled through surface 470a, away from central portion 460a of inner conduit 450a. Grooves 410a then facilitate enhanced evaporation of the repelled liquids, leaving central portion 460a of inner conduit 450a free for the passage of the exhaled breath sample. Grooves 410a are here illustrated as circumferential, however it is understood by one of ordinary skill in the art that alternative configurations of grooves 410a, such as for example those described in FIG. 2, are also applicable. Similarly, grooves 410a are here illustrated to extend along the entire length of tube 400a, however the distribution of grooves 410a may be according to any of the embodiments described above as well as other suitable distributions.

FIG. 4B schematically illustrates a breath sampling tube 400b with grooves 410b at sections 475b in an outer wall 420b and with an inner conduit 450b, according to some embodiments. Inner conduit 450b may be configured to permit gas flow along a central portion 460b of conduit 450b and to store liquids along at sections 475b. Sections 475b may be distributed at different longitudinal positions of tube 400b and may include a hydrophilic material. The liquids flowing in the inner conduit are then repelled through sections 475b, away from the central portion of inner conduit. Alternatively, sections 475b, may be distributed at different circumferential positions of tube 400b (option not shown). Alternatively, sections 475b may be distributed at different helical positions of tube 400b (option not shown). The liquids flowing in the inner conduit are then repelled through the corresponding section, away from the central portion of inner conduit.

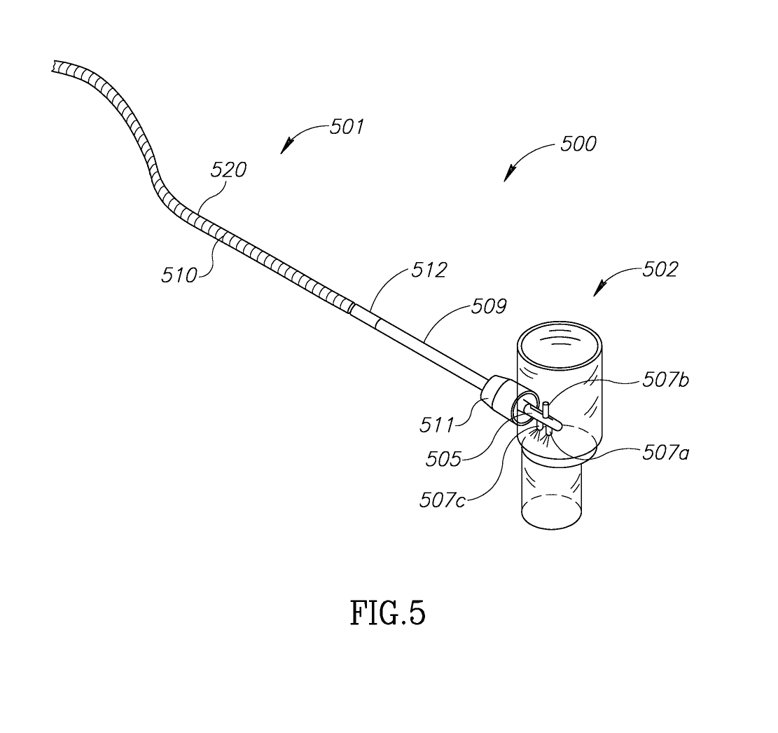

FIG. 5 schematically illustrates a breath sampling system 500 including a breath sampling tube 501 comprising a groove(s) 510 in an outer wall 520 thereof, and a connector 511, according to some embodiments. Breath sampling tube 501 may essentially correspond to any of the tubes described herein. Breath sampling system 500 is configured to enhance the evaporation rate of liquids thereby extending the life time of the tube as well as preventing liquids from reaching sensitive analyzing equipment, such as for example a capnograph (not shown). Exhaled breath sample collection is done through an airway adapter, such as airway adapter 502, which may be essentially a tube with connector fittings at each end which may be adapted to a patient airway tubing. Airway adaptor 502 may comprise at least one sampling port, such as sampling port 505, as shown. Sampling port 505 may have at least one sampling inlet, such as sampling inlets 507a-c through which the exhaled and inhaled breath sample is collected and passed into sampling system 500. The exhaled breath sample collected in airway adaptor 502 may be passed through sampling port 505 into breath sampling tube 501. Groove(s) 510 in outer wall 520 of sampling tube 501 are configured to enhance the evaporation rate of liquids from sampling tube 501, without interfering with the waveform of the sample. Optionally, sampling tube 501 may also include an inner conduit (not shown), as essentially described above. According to some embodiments, breath sampling tube 501 may be connected directly to adaptor 502 via a connector such as connector 511 (option not shown), such that the exhaled breath sample enters breath sampling tube 501 directly from breath sampling port 505 and further on to the gas analyzer (not shown). Alternatively, breath sampling system 500 may also include a moisture reduction system (MRS) 509, configured to reduce liquids from entering breath sampling tube 501. MRS 509 may at one end thereof be connected to airway adaptor 502 via connector 511 and at the other end thereof to breath sampling tube 501 via connector 512. It is understood by one of ordinary skill in the art that MRS 509 may be a specially designed tube, which may be of variable length and diameter, which may include any drying mechanism and/or material, essentially impermeable to gas, that is capable of reducing moisture level, for example Nafion®, and/or filters such as micro-porous filters or molecular sieves (material containing tiny pores that may be used to absorb moisture.

Referring to FIG. 5, the following is a description of the operation of sampling system 500 according to some embodiments. A patient is connected to a breathing apparatus or to some other ventilation means through a breathing tube or patient airway tube to which is adapted an airway adapter, such as airway adaptor 502. Samples of exhaled breath from the patient, which may include liquid secretions such as blood, mucus, water, medications, and the like, are sucked into sampling inlets, such as sampling inlets 507a-c and into sampling port 505, typically by means of negative pressure supplied by a pumping element (not shown) which may be connected to breath sampling tube 501. The breath sample (including the liquid secretions) may optionally pass from sampling port 505 into MRS 509 where moisture is extracted from the exhaled breath samples. MRS 509 is placed as close as possible to airway adaptor 502 so as to immediately try to counteract the effects of the liquids in the exhaled breath samples which may contribute to clogging in sampling tube 501. Although MRS 509 is able to extract a good portion of the moisture and liquids, significant amounts may remain in the exhaled breath samples which may hamper the accurate monitoring and analysis of the samples by the measurement sensor in addition to possibly blocking the path of the flow of the samples in sampling system 500. However, groove(s) 510 in outer wall 520 of sampling tube 501 enhance the evaporation of the liquids from the surface of tube 501 and thereby prevent liquid accumulation in the tube.

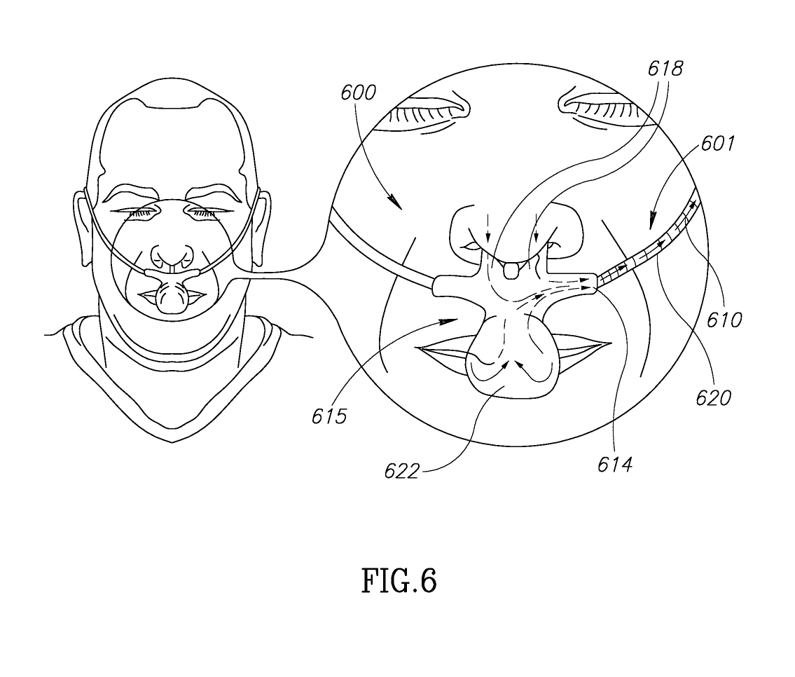

FIG. 6 schematically illustrates a breath sampling system 600 including a breath sampling tube 601 comprising a groove(s) 610 in an outer wall 620 thereof; and an oral nasal cannula, such as oral/nasal cannula 615 according to some embodiments. Breath sampling tube 601 may essentially correspond to any of the tubes described herein. Breath sampling system 600 is configured to enhance the evaporation rate of liquids thereby extending the life time of the tube as well as preventing liquids from reaching sensitive analyzing equipment, such as for example a capnograph (not shown). Breath exhaled through the subject's nose is directed through nasal prongs 618 toward an exhaled breath collection bore 614. In a similar manner, breath exhaled through the subject's mouth is collected in oral scoop element 622, and is directed to exhaled breath collection bore 614. The exhaled breath collected in exhaled breath collection bore 614 flows into breath sampling tube 601, typically by means of negative pressure supplied by a pumping element (not shown), which may be connected to breath sampling tube 601. Groove(s) 610 of sampling tube 601 are configured to enhance the evaporation rate of liquids from sampling tube 601, without interfering with the waveform of the sample.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A breath sampling tube comprising an outer wall and an inner wall, wherein said outer wall comprises at least one groove and wherein at least part of said tube is formed of a material configured to evaporate liquid.

2. The tube according to claim 1, wherein said at least one groove extends substantially along the length of said tube.

3. The tube according to claim 1, wherein said at least one groove is formed parallel to a main axis of said breath sampling tube.

4. The tube according to claim 1, wherein said at least one groove is formed orthogonal to a main axis of said breath sampling tube.

5. The tube according to claim 1, wherein said at least one groove is formed helically to a main axis of said breath sampling tube.

6. The tube according to claim 1, wherein said at least one groove is formed unevenly on said outer wall.

7. The tube according to claim 1, wherein said outer wall comprises a plurality of grooves.

8. The tube according to claim 1, wherein said at least one groove generates a rough surface in said outer wall.

9. The tube according to claim 1, wherein said at least one groove increases the surface area of said outer wall.

10. The tube according to claim 1, wherein said at least one groove enhances the evaporation of liquids from said breath sampling tube, thereby extending the life time of said breath sampling tube.

11. The tube according to claim 1, wherein said material configured to evaporate liquids is a hydrophilic material.

12. The tube according to claim 1, wherein said outer wall comprises a hydrophilic material.

13. The tube according to claim 1, wherein said inner wall comprises a hydrophilic material.

14. The tube according to claim 1, further comprising an inner conduit.

15. The tube of claim 14, wherein said inner conduit is configured to permit gas flow along a central portion of said conduit and to store liquids along a surface of said conduit.

16. The tube of claim 14, wherein the surface of said inner conduit comprises a hydrophilic material.

17. A breath sampling system comprising:
a breath sampling tube comprising an outer wall and an inner wall wherein at least part of the outer wall comprises at least one groove and wherein at least part of said tube is formed of a material configured to evaporate liquids; and
at least one connector.

18. A method comprising forming a breath sampling tube comprising an outer wall and an inner wall, wherein at least a part of the outer wall comprises at least one groove.

19. The method of claim 18, wherein at least part of the tube is formed of a material configured to evaporate water.
